(19) United States
(12) Patent Application Publication
(10) Pub. No.: US 2007/0149908 A1
(43) Pub. Date: Jun. 28, 2007

(54) Counterforce brace

(76) Inventor: Howard A. Gordon JR., Kansas City, MO (US)

Correspondence Address: CHASE LAW FIRM L.C, 4400 COLLEGE BOULEVARD, SUITE 130, OVERLAND PARK, KS, 66211, US

(21) Appl. No.: 11/709,424

(22) Filed: Feb. 22, 2007

Related U.S. Application Data
(63) Continuation-in-part of application No. 11/079,942, filed on Mar. 15, 2005.

(51) Int. Cl. A61F 5/00

(52) U.S. Cl. 602/27

(57) ABSTRACT

A stabilization brace presents a compressive, sock-like body for a compressive fit about the ankle joint and forefoot. A first pair of force straps extend from the metatarsal heads and along the opposed lateral and medial regions of the foot and ankle to offer countervailing forces therealong. First and second closure systems secure the body to the foot and offer circumferential support thereto. Alternatively, the force straps present bifurcated ends so as to extend from the metatarsal heads and heel area and along the opposed lateral and medial regions of the foot and ankle to also offer countervailing forces along the sole of the foot. The brace resists excessive supination and pronation of the foot, ankle joint and underlying structures.

This application is a continuation-in-part of application Ser. No. 11/079,942, filed Mar. 15, 2005.

### BACKGROUND OF THE INVENTION

This invention relates to stabilization braces and more particularly to a support which stabilizes the ankle and forefoot of the user at a desired neutral position.

The supination of a foot combines inward rotation at the ankle, adduction of the hind foot inversion of the forefoot and medial arch elevation. Basically, supination produces a medial (inward) movement at the talocalcaneal (subtalus) joint known as inversion as well as similar movement between the hind foot (talus) and mid foot (navicular) bones. A foot that supinates appears to naturally favor the lateral region or outside edge of the foot. A certain amount of supination is normal during the toe off stage of running, i.e., when the heel first lifts off the ground until the end of the step. During excessive supination, the lateral structures of the foot tighten which may rupture or avulse portions of the lateral collateral ligaments resulting in a “rolling over” of the ankle or ankle sprain.

The pronation of a foot produces opposite foot behavior of the subtalar joint including adduction of the forefoot, eversion of the hind foot and dorsiflexicon. Basically, pronation produces a lateral (outward) movement of the foot at the subtalar joint known as eversion as well as similar hind foot and mid foot movements. A foot that pronates appears to naturally favor the inside or medial edge of the foot. The foot naturally pronates to absorb the shock resulting from the heel hitting the ground. Excessive pronation increases the stress on the inside/medial portion of the foot which may lead to various injuries such as arch strain, heel pain, inner ankle pain and shin splints.

Accordingly, it is desirable to preclude excessive pronation and/or supination of the ankle joint and foot. Thus, various devices have been proposed to prevent ankle and foot injuries by attempting to maintain the foot in a “neutral position”. Although assumably effective in their operation, such devices have failed to simultaneously provide the effective stabilization of the ankle and forefoot and in a neutral position. This desired stabilization, which resists excessive pronation and/or supination, positions the foot for a proper impact during walking, running and jumping rather than on the edge of the foot while in an excessive pronated or supinated position.

In response thereto, stabilization braces are presented for simultaneously maintaining the ankle and the forefoot at a neutral position while permitting natural movement through a full range of motion. A first brace presents an elastic, sock-like body positioned in a compressive fit about the ankle and foot of the wearer. A pair of torque straps is anchored to the body adjacent the distal (head) ends of the first and fifth metatarsals. Each strap is then wound about the forefoot, heel and ankle and anchored above the ankle joint so as to present countervailing forces along the foot and ankle joint complex. These forces resist excessive movement of the foot and ankle from their normal, neutral position without precluding normal ambulatory movement thereof. The brace design anatomically fits either foot about the heel, ankle and forefoot aspects thereof. A first closure system which allows the sock-like body to be easily slipped onto the foot. A second closure system maintains the circumferential integrity of the brace about the ankle joint region and presents anchor zones for the free ends of the bilateral torque straps. This structure allows the user to variably adjust the tension on these torque straps and thus the countervailing forces presented thereby.

A second brace similar in construction presents bifurcated ends at the end of each torque strap. These ends are respectively anchored adjacent the distal ends of the metatarsals, Achilles tendon and the heel. Each strap is then wound about the foot as above with the dual anchor zones enhancing the distribution of forces along the foot.

It is therefor a general object of the invention to provide a brace which simultaneously secures the ankle and the forefoot at a neutral position so as to resist excessive supination and pronation thereof.

Another object of the invention is to provide a brace, as aforesaid, which presents a pair of torque straps which present countervailing forces along the ankle and foot regions extending between the metatarsal heads and the superior region of the ankle joint complex to resist excessive pronation or supination.

A particular of this invention is to provide a brace, as aforesaid, which presents a pair of torque straps either anchored at its ends to a zone adjacent the metatarsals or to spaced-apart zones adjacent the metatarsals and heel of the wearer.

A further object of this invention is to provide a brace, as aforesaid, having a sock-like body which slips over the foot and provides a compression fit thereto between the metatarsal heads and the superior region of the ankle joint complex.

Another object of this invention is to provide a brace, as aforesaid, wherein a first closure system allows the brace to be used with variously sized feet while maintaining a compressive fit.

A still further object of this invention is to provide a brace, as aforesaid, having a second closure mechanism for providing circumferential forces about the ankle joint complex while presenting anchor points for the torque straps.

Another particular object of this invention is to provide a brace, as aforesaid, which presents a pattern to fit the ankle, heel and forefoot aspects of the foot of the wearer.

A more particular object of the invention is to provide a brace as aforesaid, which uses either heat seals along the seams of the brace pattern in lieu of stitching so as to enhance the fit of the brace and/or stitching.

Other objects and advantages of this invention will become apparent from the following description taken in connection with the accompanying drawings, wherein is set forth by way of illustration and example, a now preferred embodiment of this invention.

### BRIEF DESCRIPTION OF THE DRAWINGS

FIG. 1 is a top/dorsal view of the right foot of the wearer and showing in phantom lines the metatarsals and phalanges of the foot of the wearer;

FIG. 2 is a perspective view of the right foot of the wearer showing the sock-like body thereon, the unwrapped countervailing force straps (fragmentarily shown), the first closure system prior to full engagement and the second closure system disengaged from the ankle joint complex;

FIG. 3 is a view, as in FIG. 2, showing the FIG. 2 closure system in an engaged/locked position;

FIG. 4 is a view as in FIG. 3 showing the initial wrapping of the medial torque strap about the dorsal aspect of the foot, underneath the heel and along the medial side of the ankle joint complex prior to engagement of the free end of the medial strap to a superior position above the ankle joint complex;

FIG. 5 is an opposed perspective view of the foot in FIG. 4 showing the initial wrapping of the lateral torque strap across the dorsal aspect of the foot;

FIG. 6 is a view of the FIG. 5 foot showing the continuing engagement of the lateral strap across the dorsal aspect of the foot, underneath the heel extension of the lateral strap along the lateral side of the ankle joint complex prior to engagement of the free end of the lateral strap to a superior position above the ankle joint complex;

FIG. 7 is a bottom view of the foot showing the metatarsal anchor zone of the straps adjacent the free edge of the body and the extension of the straps wound under the heel;

FIG. 8 is a lateral view of the foot with brace secured thereon;

FIG. 9 is a view of the pattern of the brace showing the tongue element;

FIG. 10 is a view of the pattern of the brace showing the portion for encompassing the ankle joint;

FIG. 11 is a view of the pattern of the brace showing the portion for encompassing the heel and forefoot;

FIG. 12 is a view of one of the counter force straps;

FIG. 13 is a diagrammatic view showing pronation and supination of the right foot from the rear thereof;

FIG. 14 is a bottom view of the foot showing an alternative embodiment of the countervailing force straps presenting bifurcated ends anchored adjacent the metatarsals and heel of the wearer;

FIG. 15 is a perspective view of the wearer showing the sock-like body thereon and the initial wrapping of the medial strap about the dorsal aspect and underneath the foot of the wearer; and

FIG. 16 is a lateral view of the foot with the alternative embodiment of the brace secured thereon.

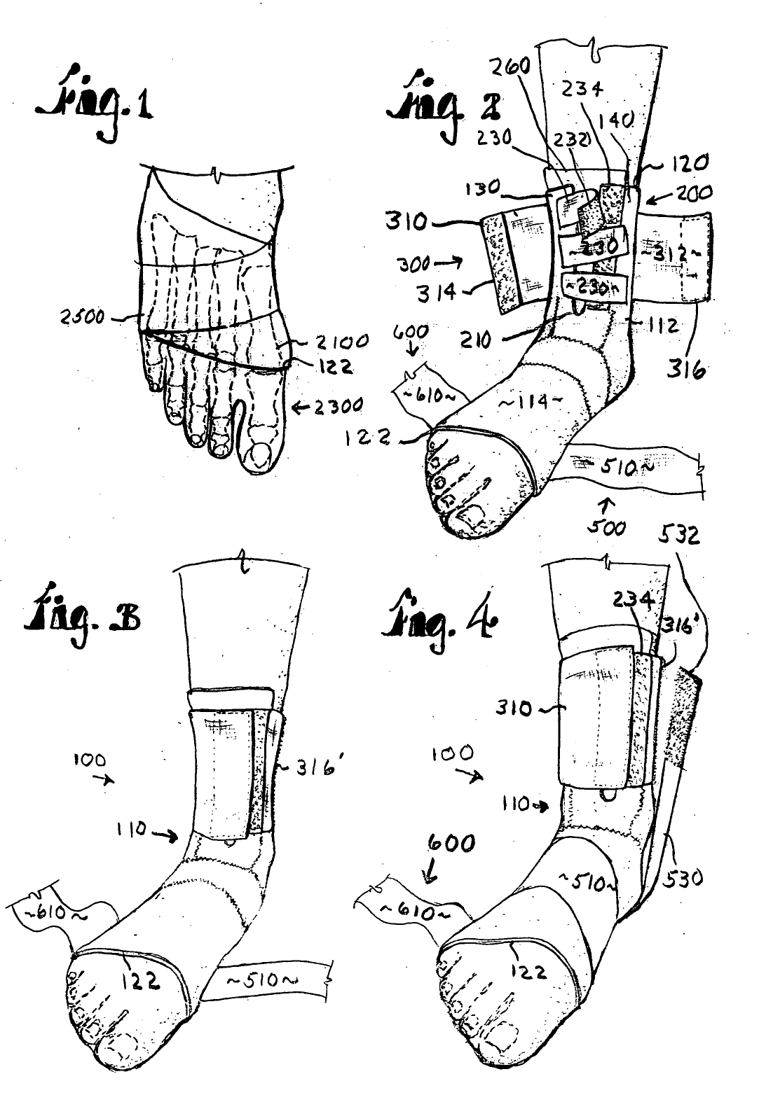

### DESCRIPTION OF THE PREFERRED EMBODIMENT

Turning more particularly to the drawings, the first brace 100 comprises a sock like body 110 made of an elastic material. The body 110 presents first 112 and second 114 integral sleeves made from joined blanks 112′, 114′. The body 110 provides a compression fit about the ankle joint complex and forefoot of the wearer. The forefoot is described as that region of the foot extending from the heel to approximate the distal/head ends of the first 2100 and fifth metatarsals 2500. The body 110 presents a first free edge 120 which circumscribes the foot at a superior position above the ankle joint complex. A second free edge 122 circumscribes the foot adjacent the distal ends/heads of the first 2100 and fifth 2500 metatarsals. It is preferred that this edge 122 not extend beyond such distal ends as it may present discomfort to the foot during flexion. As such, upon slipping the body 110 over the foot, the body 110 presents a compression fit between these edges 120, 122.

The initial extension of the foot through the opening presented by the free edge 120 is enhanced by a closure system 200. This closure system is presented by a U-shaped notch 210 which extends from the free edge 120. As such, first and second flaps 130, 140 are presented which allow the user to increase the initial opening presented by the upper free edge 120. As such, a foot of various sizes may be initially extended through this free edge 120 for ultimate extension of the phalanges 2300 through the downstream free edge 122. Aperture 190 allows for extension of the heel therethrough.

The closure system 200 presents a plurality of tabs 230 which are secured at one end along the longitudinal extent of the notch 210 on flap 130. These tabs have Velcro™ fastener material 232 on the inside thereof. Complementary Velcro™ material 234 is located along the extent of the notch 210 on the opposed flap 140. The engagement of each tab 230 fastener 232 to the complementary fastener 234 enables the user to adjust the compression of the sleeve 112 about the ankle joint region as the degree of overlap of these fasteners 232, 234 can be selectably adjusted. A tongue 260 is attached to the interior of the body portion 112 for extension along notch 210 so as to preclude the sleeve 112 and tabs 230 from directly contacting the user.

Further attached to the rear of body portion 112 adjacent edge 120 is a second closure system 300 which presents first 310 and second 312 straps made of an elastic and/or non-stretchable material. The straps 310, 312 present complementary Velcro™ fasteners 314, 316 at the free ends thereof. Accordingly, these flaps are wrapped circumferentially around the ankle joint complex and attached one to the other as shown in FIG. 3. The degree of overlap of the complimentary fasteners 314, 316 adjusts the compressive fit of straps 310, 312 about the ankle joint and thus maintains the circumferential integrity of the brace about the ankle joint complex. These straps 310, 312 further present Velcro™ fasteners 316, 316′ along the lateral and medial sides of the ankle joint which mate with complementary Velcro™ fasteners at the ends of the bilateral torque straps as to be further described.

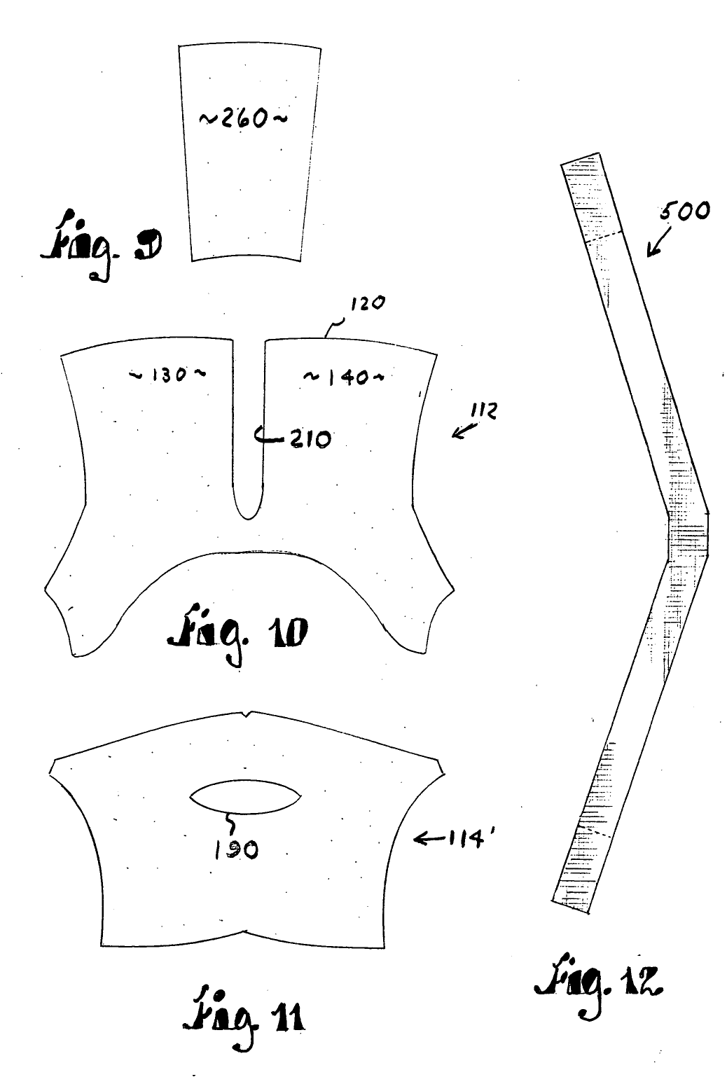

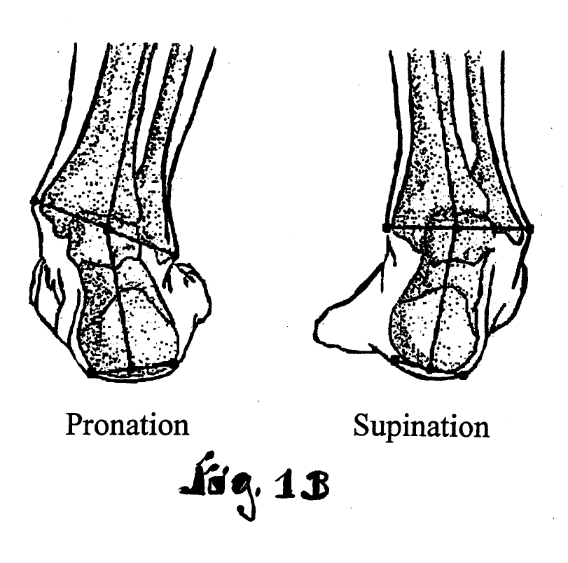

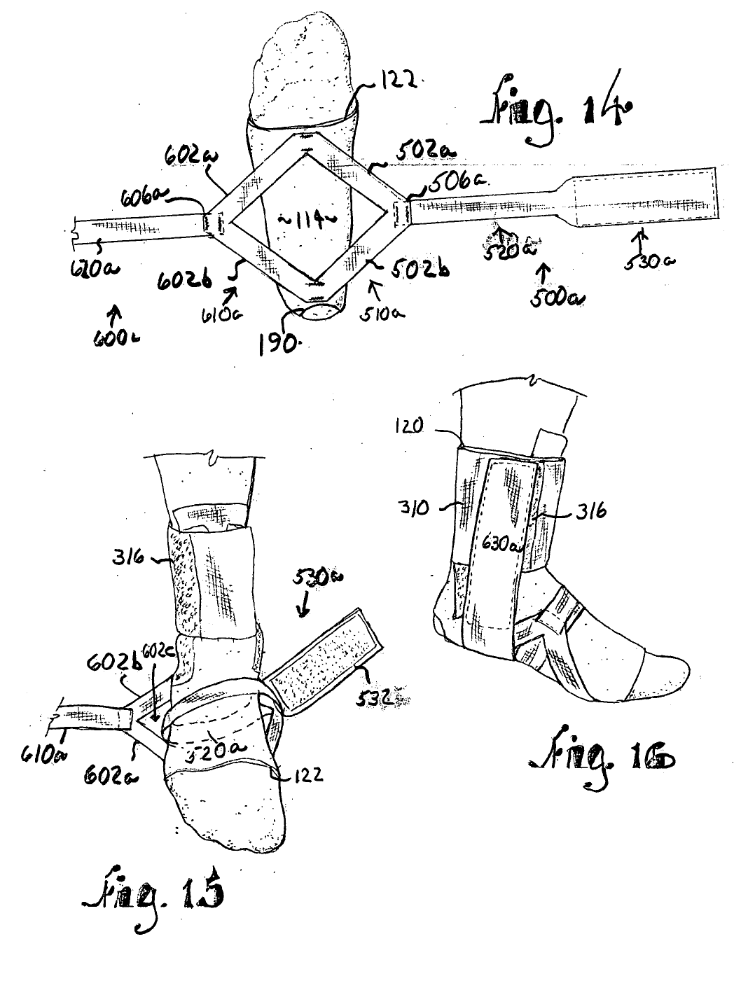

The brace 100 further includes medial 500 and lateral 600 countervailing force straps which are anchored at a location 550 underneath the sleeve 114 and adjacent the free edge 122 (FIG. 7). These straps, 500, 600 are preferably made of a non-elastic material. It is understood that the straps 500, 600 may be a single elongated strap anchored at 550 so as to present the medial 500 and lateral 600 straps or separate straps extending therefrom.

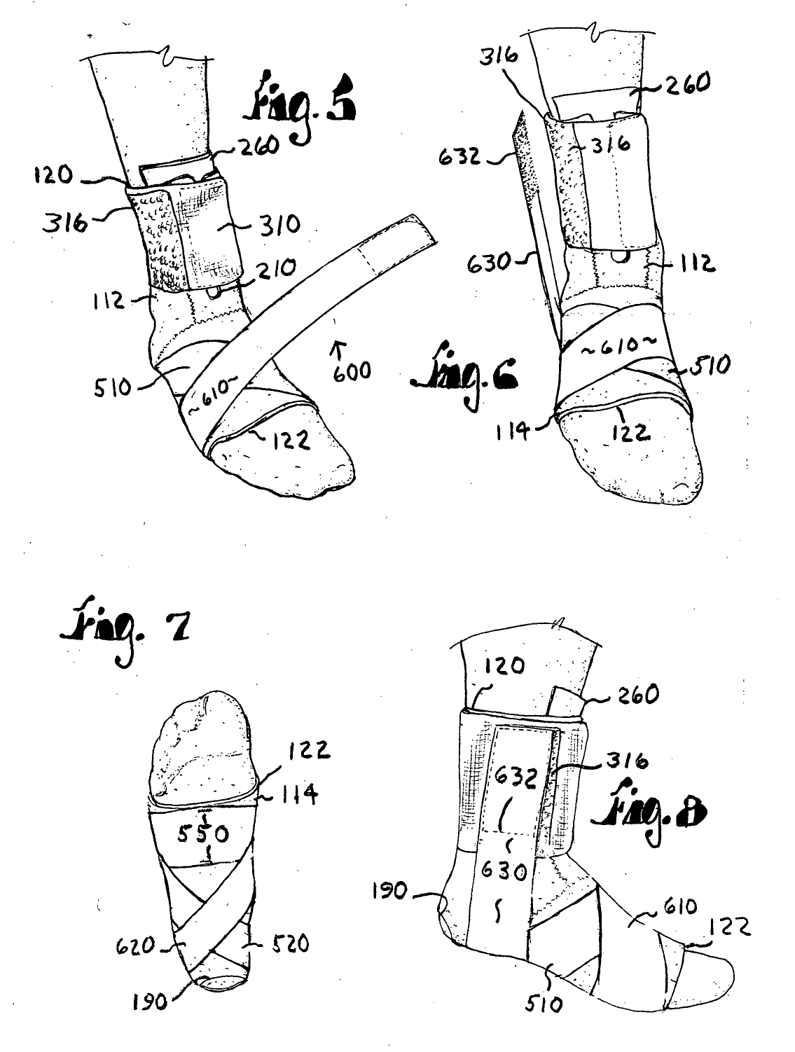

Accordingly, the right foot being shown, the medial strap 500 is first wrapped so as to provide a force along the forefoot, heel and ankle regions of the wearer. As such, a first extension 510 of strap 500 extends from zone 550 and to the medial side of the foot, across the dorsal aspect of the forefoot and rearwardly towards the lateral side of the heel. A second extension of the strap 520 is wound underneath the heel as best shown in FIG. 7. Subsequently, the remaining portion of the strap 530 is then brought upwardly along the medial side of the ankle joint region as best shown in FIG. 4. The end of the strap 500 presents Velcro™ material 532 designed to mate with the Velcro™ 316′ presented on flap 312 of the closure system 300. As such, the wearer by pulling on strap 500 at extension 530 can variably adjust the mating of the strap 550 end to this Velcro™ mating material 316′ and thus adjust the tension along the medial strap 500. In turn this tension is transmitted as a force along the medial and dorsal aspects at the foot, the lateral side of the heel, underneath the heel and along the medial side of the heel and ankle joint complex.

Subsequently, a first extension 610 of the lateral strap 600 is wound about the dorsal aspect of the foot from the lateral side thereof, across the dorsal aspect of the forefoot and rearwardly towards the medial side of the heel (FIGS. 5, 6). A second strap extension 620 is then wound underneath the heel (FIG. 7) with a third extension 630 brought upwardly along the lateral side of the heel and ankle joint (FIG. 8). The end of the strap extension 630 likewise presents a Velcro™ fastener 632 which engages complementary material 316 presented on strap 310 of the closure system 300. Again, the variable anchoring of the strap extension 630 to this Velcro™ material 316 enables the user to adjust the tension along this strap 600. In turn this tension is transmitted as a force, opposite that force presented by strap 500, along the lateral and dorsal aspects at the foot, the medial side of the heel, underneath the heel and along the lateral side of the ankle joint complex.

Accordingly, the body 110 along with the closure systems 200, 300 provides compressive forces about the ankle and foot of the user. The medial 500 and lateral 600 straps present countervailing forces beginning at the metatarsal heads, along the forefoot and upward along the ankle joint complex. As previously discussed, pronation and supination of the foot respectively present opposed movements at the subtalar joint, hind foot and mid foot such that the foot favors the lateral region foot (supination) or the medial region (pronation) of the foot. Straps 500, 600 thus present countervailing forces in these regions so as to resist movement of the ankle and foot, inclusive of its underlying structures, from excessive pronation or supination. Thus, as the foot is held in a neutral position, the probability of the user landing on an excessively supinated or pronated foot during walking, jumping and/or running is reduced which reduces the probability of injury. The use of these counter force straps 500, 600 with or without the compressive fit offered by the body 110 and the compression mechanisms 200, 300 enhances the prophylactic advantages of the brace 100.

Alternatively, a second embodiment of brace 100a is shown in FIGS. 14-16. Brace 100a presents alternative medial 500a and lateral 600a force straps. The end of the straps 500a, 600a present bifurcated ends 502a, 502b, 602a, 602b. The strap ends 502a, 602a are anchored underneath the sleeve 114 adjacent the free edge 122 and adjacent the metatarsals. The ends 502b, 602b are anchored underneath the sleeve 114, adjacent the edge presented by heel aperture 190 and thus underneath the heel. These bifurcated ends are included in a first extension 510a, 610a of each strap and function as the first extensions 510, 610 explained hereinabove. Subsequent extensions 520a, 620a, 530a, 630a with Velcro™ material thereon function as above described in embodiment 100.

Embodiment 100a is utilized in a manner as above described in embodiment 100. As such, the foot is initially extended through the opening 120 presented by the free edge of the body member 110 for ultimate extension of the metatarsal flanges 2300 through this free edge and the downstream free edge 122 with extension of the heel through aperture 190. First and second closure systems 200 and 300 function as above described.

The medial strap 500a is first wrapped so as to provide a force along the forefoot, heel and ankle regions of the wearer. As such, the free end of the strap 500a is first extended through the opening 602c presented by the bifurcated strap ends 602a, 602b of lateral strap 600a. The first extension 510a of strap 500a, comprising, in the main, ends 502a, 502b, extend from the two spaced anchor points on sleeve 114 to the medial side of the foot across the dorsal aspect of the forefoot and rearwardly towards the lateral side of the foot.

A second extension 520a of the strap comprising, in the main, the reduced lateral portion of strap 520a is wound underneath the foot and shown in phantom in FIG. 15. The remaining portion of the strap 530a is brought along the medial side of the ankle joint strap such that the end of the strap can mate the Velcro™ material 532 thereon with the Velcro™ material 316′ presented on the flap 312 of the closure system. As above, the wearer by pulling on the strap 500a at extension 530a can adjust the mating of the strap 530a to the Velcro™ 316′ and thus the tension along the medial strap 500a. This tension is transmitted along the medial dorsal aspects of the foot, the lateral side of the heel, the medial side of the heel and ankle joint and along the underneath of the heel as presented by the dual anchor points of the bifurcated strap ends 502a, 502b.

Strap 600a works on foot in an opposed manner to present countervailing forces on the foot. Thus, the first extension 610a of the lateral strap 600a including, in the main, the bifurcated straps 602a, 602b, is wound about the dorsal aspect of the foot from the lateral side thereof, across the dorsal aspect of the foot and rearwardly toward the medial side of the heel. A second strap extension 620a, i.e., primarily the reduced lateral portion, is then wound underneath the heel with a third extension 630a brought upwardly along the lateral side of a heel and ankle joint complex (FIG. 16). The end of the strap 630a likewise presents a Velcro™ fastener which engages complementary material 316 presented on strap 310 of closure system 300. The variable Velcro™ anchoring of this strap extension 630a allows users to adjust the tension of the strap 600a which is transmitted as a force opposite that presented by strap 500a in a manner as described in embodiment 100. Due to the bifurcated strap ends 602a, 602b and dual anchor zones, forces are presented along the underneath of the foot. Accordingly, I have found that such a brace 100a presents an enhanced leverage along all aspects of the foot.

It is also noted that the bifurcated ends of one of the straps, e.g., 602a, 602b, present a guide 602c for the initial extension of the free end of the opposite strap, e.g., 500a, so as to direct the initial winding of the first strap, e.g., 500a, in a proper direction. Thus, the proper application of the countervailing force straps 500a, 600a about the foot of the wearer is assured.

It is noted that embodiment 100a is similar in construction to embodiment 100. Thus, identical numbers refer to identical elements as utilized between the two braces 100, 100a.

Although now preferred embodiments of this invention have been described herein, it is understood that such a description is not limited except as set forth in the claims and allowable functional equivalents thereof.

Having thus described the invention, what is claimed as new and desired to be secured by Letters Patent is as follows:

1. A stabilizing brace comprising:
a body of material having a sock-like configuration adapted to encompass an ankle joint and a portion of a foot of a wearer, said body presenting:
a first free edge adapted for encompassing the ankle of the wearer at a superior location above the ankle joint, said first free edge presenting a first aperture for initial extension of a wearer's foot therethrough;
a second free edge presenting a second aperture adapted for extension of the wearer's foot therethrough, said second free edge adapted for encompassing the foot of the wearer approximate the distal ends of the metatarsals of the foot upon said extension of the wearer's foot therethrough, said body extending between said free edges;
a first elongated strap presenting first and second bifurcated ends, said first bifurcated end anchored at one end at said second free edge of said body with said second bifurcated end anchored to said body at a position displaced from said first bifurcated end, said first strap comprising:
a first extension including said bifurcated ends adapted for medially extending from said anchored ends and across a dorsal aspect of the foot to a lateral position on a wearer's heel;
a second integral extension adapted for extending from the lateral position of the heel, underneath the heel and to a medial side of the heel;
a third integral extension adapted for extending from the medial side of the heel and upwardly along a medial side of the ankle joint to a superior location thereabove;
first means for attaching a portion of said third extension of said first strap to said body at the superior position above the ankle joint;
a second elongated strap presenting first and second bifurcated ends, said first bifurcated end of said second strap anchored at one end at said second free edge of said body with said second bifurcated end of said second strap anchored to said body at a position displaced from said bifurcated end of said second strap, said second strap comprising:
a first extension adapted for laterally extending from said anchored end and across the dorsal aspect of the foot to a medial position on the wearer's heel;
a second integral extension adapted for extending from the medial side of the heel, underneath the heel and to a lateral side of the heel;
a third integral extension adapted for extending from the lateral side of the heel and upwardly along a lateral side of the ankle joint to a superior location thereabove;
second means for attaching a portion of said third extension of said second strap to said body at the superior position above the ankle joint, said first and second straps adapted to present countervailing forces along the foot and ankle joint to resist excessive supination and pronation thereof.

2. The brace as in claim 1 wherein said first attaching means comprises:
a first fastener element on said third extension of said first strap;
a second complementary fastener element on said body at a superior position above the ankle joint, said engagement of said first and second fastener elements attaching said third extension of said first strap to said body.

3. The brace as in claim 2 wherein said second attaching means comprises:
a third fastener element on said third extension of said second strap;
a fourth complimentary fastener element on said body at a superior position above the ankle joint, said engagement of said third and fourth fastener elements attaching said third extension of said second strap to said body.

4. The brace as in claim 1 wherein said bifurcated ends of said first and second straps are adapted to be anchored to said body adjacent a sole of the wearer.

5. The brace as in claim 1 further comprising:
a notch in said body portion extending from said first free edge, said notch presenting first and second flaps, each flap including a portion of said first free edge, a manipulation of said flaps adjusting a size of said first aperture presented by said first free edge.

6. The brace as in claim 5 further comprising:
means for maintaining said flaps in place about the ankle joint of the wearer.

7. The brace as in claim 5 further comprising:
a tongue spanning said notch.

8. The brace as in claim 1 further comprising:
a third strap having an end attached to said body at a position adjacent said first free edge;
a fourth strap having and end attached to said body at a position adjacent said first free edge, said third and fourth straps adapted to encircle the ankle joint region of the wearer; and
means for engaging said third and fourth straps.

9. The brace as in claim 8 wherein said engaging means comprises:
a fastener at a free end of said third strap;
a complementary fastener element at a free end of said fourth strap, said fastener elements on said third and fourth straps engageable one to the other to regulate a degree of said support offered by said third and fourth straps about the ankle joint.

10. The brace as in claim 5 further comprising:
a third strap attached to said body at a position adjacent said first free edge;
a fourth strap attached to said body at a position adjacent said first free edge, an engagement of said third and fourth straps adapted to cover said notch and encircle the ankle joint to offer support thereto.

11. The brace as claimed in claim 10 further comprising:
a fastener at a free end of said third strap;
a complementary fastener element at a free end of said fourth strap, said fastener elements on said third and fourth straps variably engageable one to the other to provide said engagement of said third and fourth straps about the ankle joint.

12. A support brace comprising:
a body made of a material adapted to extend between a region above a wearer's ankle joint and the heads of a wearer's metatarsals upon placement of a wearer's foot therein;
a first strap having bifurcated ends adapted to be respectively secured to the body at regions adjacent the underside of the metatarsals and a heel of the wearer, said first strap adapted for medially extending from said regions across the dorsal portion of the foot to a lateral side of the heel, underneath the heel and along a medial side of the ankle joint for upward extension therealong, said first strap having a free end;
first means for engaging said free end of said first strap at a selectable position above the ankle joint;
a second strap having bifurcated ends adapted to be respectively secured to the body at regions adjacent the underside of the metatarsals and the heel, said second strap adapted for laterally extending from said regions across the dorsal portion of the foot to a medial side of the heel, underneath the heel and along a lateral side of the ankle joint for upward extension therealong, said second strap having a free end;
second means for engaging said free end of said second strap at a selectable position above the ankle joint, said first and second straps presenting countervailing forces along the foot and ankle joint so as to resist excessive supination and pronation.

13. The brace as in claim 12 wherein said first engaging means presents a plurality of selectable areas of engagement for said free end of said first strap, whereby to adjust tension in said first strap.

14. The brace as in claim 13 wherein said second engaging means presents a plurality of selectable areas of engagement for said free end of said second strap, whereby to adjust tension in said second strap.

15. The brace as in claim 12 further comprising:
means on said body at said edge for adjusting a size of said body at the region above the ankle joint whereby to enhance an initial protrusion of a wearer's foot into said body.

16. The brace as in claim 15 further comprising:
means on said body adapted for presenting adjustable compressive forces about the ankle joint region.

17. The brace as in claim 12 further comprising:
third and fourth straps on said body adapted to encircle the ankle region;
means for attaching said free ends of said third and fourth straps one to the other in a manner to adjust the degree of support presented by said third and fourth straps about the ankle joint region.

18. A stabilizing brace comprising:
a body of material adapted to encompass an ankle and a portion of the foot of a wearer, said body presenting:
a first opening adapted for initial extension of a wearer's foot therethrough;
a second downstream opening adapted for extension of the metatarsal heads of a wearer's foot therethrough, said second opening presenting an edge encompassing the foot of the wearer adjacent the metatarsals heads of the foot and displaced from the heel of the wearer upon extension of the wearer's foot therethrough;
means for regulating a size of said first opening to enhance the initial extension of the wearer's foot therethrough;
a first elongated strap having first and second bifurcated ends with said first bifurcated end attached at said second free edge of said body and said second bifurcated end attached to said body at a position adjacent the wearer's heel, said first strap adapted to extend across the dorsal aspect of the foot from the medial to the lateral side thereof, underneath the heel and along the medial side of the ankle from the heel to a superior position above the ankle;
first means for attaching said free end of said first strap to a selectable, superior position above the ankle;
a second strap having first and second bifurcated ends with said first bifurcated end of said second strap attached at said second free edge of said body and said second bifurcated end of said second strap attached to said body at a position adjacent the wearer's heel and a free end, said second strap adapted to extend across the dorsal aspect of the foot from the lateral to the medial side thereof, underneath the heel and along the lateral side of the ankle from the heel to a superior position above the ankle;
second means for attaching said free end of said second strap to a selectable position above the ankle joint, said first and second straps about the foot presenting countervailing forces thereto whereby to resist excessive supination or pronation of the foot or both.

19. A brace as in claim 18 further comprising:
at least one third strap encircling said body at a region adjacent said first opening said third strap adapted to encircle the ankle joint of the wearer in a compressive fit therearound.

20. The brace as claimed in claim 18 wherein said bifurcated ends of said first or second straps, or both, are adapted to be attached to said body at said positions adjacent the underside of the foot.
